(19) United States
(12) Patent Application Publication
(10) Pub. No.: US 2012/0292252 A1
(43) Pub. Date: Nov. 22, 2012

(54) TUBULAR SURFACE COALESCERS

(76) Inventors: George Chase, Wadsworth, OH (US); Goutham Viswanadam, Cuyahoga Falls, OH (US); Barry Mark Verdegan, Stoughton, WI (US); Saru Dawar, McFarland, WI (US); Mark T. Wieczorek, Cookeville, TN (US)

(21) Appl. No.: 13/470,766

(22) Filed: May 14, 2012

Related U.S. Application Data
(60) Provisional application No. 61/487,985, filed on May 19, 2011.

(51) Int. Cl. B01D 29/13; B01D 46/00; B01D 11/04; B01D 29/52; C07C 7/144

(52) U.S. Cl. 210/634; 210/497.01; 210/497.1; 210/323.2; 585/818; 95/273

(57) ABSTRACT

Disclosed are tubular surface coalescers, systems, and methods for coalescing a mixture of two phases, namely a continuous phase and a dispersed phase. The disclosed tubular surface coalescers, systems, and methods include or utilize one or more layers of media material having a distinct mean pore size and wettability applied to a surface of a porous tubular support structure.

### CROSS-REFERENCE TO RELATED APPLICATIONS

The present application claims the benefit of priority under 35 U.S.C. §119(e) to U.S. Provisional Application No. 61/487,985, filed on May 19, 2011, the content of which is incorporated herein by reference in its entirety.

### BACKGROUND

The field of the invention relates to coalescers, coalescing media material, coalescer elements, coalescing systems, and coalescing methods for coalescing a mixture of two phases, namely a continuous phase and a dispersed phase. In particular, the field relates to tubular surface coalescers, systems, and methods for coalescing drops of the dispersed phase in order to collect and remove the dispersed phase from the mixture.

Coalescers are widely used to remove immiscible droplets from another liquid or gaseous phase. For example, high pressure common rail (HPCR) fuel filtration applications require removal of essentially all non-dissolved water from ultra-low sulfur diesel (ULSD) fuel and biodiesel. These ULSD fuels tend to have lower interfacial tensions, and therefore contain smaller size water drops and form more stable emulsions than previous diesel fuel. This presents a challenge in coalescing and removing these smaller size water drops.

Mixtures of a continuous phase and a dispersed phase also often contain surfactants which further compounds coalescence of the dispersed phase, because the rate of coalescence between drops of the dispersed phase is reduced by the presence of surfactants. Furthermore, surfactants may adversely affect performance of a coalescer by modifying the surface properties of filter media used in the coalescer. For example, in fuel water separators (FWS), the presence of surfactants may render the filter media more hydrophobic and less wettable to water drops dispersed in hydrocarbon fuel. Coalescers that provide high levels of droplet separation under such conditions are needed.

In the case of coalescers utilized in crankcase ventilation (CV) filter applications, near 100% removal efficiency of oil mist is required to protect the turbocharger in closed CV applications and to protect the environment in open CV applications. This is challenging because CV applications may include oil droplets in the submicron size range. Similar performance challenges exist in natural gas, water, hydraulic, coolant and machine fluid applications.

Coalescers also should occupy minimal volume in a given application. Existing coalescers use formed or pleated cylindrical tube configurations, which tend to be relatively large and occupy significant volume. It is desirable for coalescers to utilize smaller volumes or to be adaptable to form different shapes in some applications. In summary, an improved coalescer is needed that yields high performance, robust performance in the presence of surfactants, and flexibility in packaging. This need is not being met by current technology.

### SUMMARY

Disclosed are coalescer elements, coalescing media material, coalescing systems, and coalescing methods for coalescing a mixture of two immiscible phases, namely a continuous phase and a dispersed phase. The disclosed coalescing element, coalescing media material, elements, systems, and methods may be utilized in order to remove or collect the dispersed phase from the mixture and may comprise or consist of a single layer or multiple layers of coalescing media material applied to an outer surface of a porous tubular support structure.

The disclosed coalescer elements typically are surface coalescer elements which include one or more layers of coalescing media material applied to an outer surface of a porous tubular support structure. As such, the disclosed coalescer elements may be referred to as “tubular surface coalescers.”

In the disclosed tubular surface coalescers, the porous tubular support structure may have a suitable, relatively small diameter (e.g., in some embodiments less than 40 mm, 30 mm, 20 mm or 10 mm, and in some embodiments a diameter between about 1 mm and about 10 mm). The tubular support structures are chosen to have gaps or apertures and the like, the gaps or apertures being spanned by one or more layers of coalescing media material such that the mixture of immiscible materials can be forced to flow in a direction through the coalescing media material, with the dispersed phase being substantially blocked by the coalescing media material and the continuous phase passing therethrough and through the gaps or apertures of the tubular support structure. Suitable support structures may include, but are not limited to, springs such as compression springs having a suitable average distance between coils (e.g., an average distance between coils of about 0.5 mm to about 2.5 mm), a mesh or trellis material that is formed into a tubular support structure, a tubular structure formed from lateral ribs and peripheral rings that support the lateral ribs, porous hollow tubes (e.g., a perforated tube), and a foam tube (e.g., a polyurethane or polyether foam tube which optionally may be hollow), all of which may be formed, for example, from polymeric or ceramic material. The diameter of the porous tubular support structure may be selected based on the average diameter of dispersed droplets in a continuous phase which for which the tubular surface coalescer is configured to remove from the continuous. For example, the ratio of the diameter of the porous tubular support structure to the average diameter of dispersed droplets to be removed in some embodiments may be less than about 100, 50, 30, 20, or 10.

The length of the disclosed tubular surface coalescers may vary. However, typically the length of the disclosed tubular surface coalescers is significantly greater than the diameter of the disclosed tubular surface coalescers. For example, in some embodiments the ratio of the length of the disclosed tubular surface coalescers to the diameter of the disclosed tubular surface coalescers (i.e., the aspect ratio) is at least about 5, 10, 20, 50, 100, or greater.

In the disclosed tubular surface coalescers, the one or more layers of coalescing media material applied to the outer surface of the porous tubular structure may comprise fibers having a suitable diameter. In some embodiments, the fibers have a mean diameter between about 0.05 μm and 5 μm. Suitable fibers may include, but are not limited to, polymeric fibers, ceramic fibers, and carbon fibers. The fibers may be applied to the outer surface of the porous tubular structure by methods including, but not limited to, electro-spinning, melt-spinning, or melt-blowing.

In the disclosed tubular surface coalescers, the one or more layers of coalescing media material applied to the outer surface of the porous tubular structure may have suitable physical characteristics such as pore size. In some embodiments, the tubular surface coalescers disclosed herein include one or more layers of coalescing media material having a mean pore size M, wherein 0.2 μm≦M≦12.0 μm. In further embodiments, the tubular surface coalescers disclosed herein include one or more layers of coalescing media material having a maximum pore size M_{M} where 1≦M_{M}/M≦3.

The disclosed tubular surface coalescers may include at least two adjacent layers of coalescing media material applied to the outer surface of the porous tubular support structure, namely an outer first layer of media material and an inner second layer of media material, where the first layer and the second layer having mean pore sizes M₁ and M₂, respectively, and M₁>M₂. In some embodiments, M₁ is at least about 2.5 times greater than M₂ and in some embodiments M₁≧30 μm. In further embodiments, 0.2 μm≦M₂≦12.0 μm.

The disclosed tubular surface coalescers may include at least two adjacent layers of coalescing media material applied to the outer surface of the porous tubular support structure, namely an outer first layer of media material and an inner second layer of media material, where the outer first layer of media material comprises relatively coarse fibers (e.g., fibers having an average fiber diameter of about 10 μm to about 100 μm) and the inner second layer of media material comprises relatively fine fibers (e.g., fibers having an average fiber diameter of about 0.05 μm to about 5 μm. In some embodiments, the coalescing media material is formed by electro-spinning, melt-spinning, or melt-blowing the inner second layer of media material, and subsequently electro-spinning, melt-spinning, or melt-blowing the outer first layer of media material.

The disclosed tubular surface coalescers may be flexible or bendable, and optionally may be elastic. In some embodiments, the disclosed tubular surface coalescers may be bent at a 90° angle or a 180° angle. The disclosed tubular surface coalescer also may form superstructures such as a coiled tube or an undulating tube.

The disclosed tubular surface coalescers may be contained in a housing, such as a housing having an upstream inlet structured to receive a mixture of a continuous phase and a dispersed phase and a downstream outlet structured to discharge the mixture after coalescing of the dispersed phase, wherein the continuous phase will have a reduced amount of dispersed phase, or if 100% coalescence and removal of the dispersed phase is achieved then to discharge the continuous phase. Typically, the housing may include a drain for releasing the coalesced dispersed phase. The disclosed tubular surface coalescers may be mounted in the housing with their ends sealed, for example, via mounting the ends of the coalescers in a polymeric material such as polyurethane.

Also disclosed are coalescing systems that include or utilize the disclosed tubular surface coalescers. For example, where the coalescing systems comprise one or more of the surface coalescers and the surface coalescers are modular. In some embodiments, the coalescing systems comprise one or more of the disclosed coalescers aligned in parallel.

In some embodiments, the disclosed systems are configured for removing a dispersed phase (e.g., a liquid phase) from a mixture comprising the dispersed phase in a continuous phase (e.g., another liquid phase or a gaseous phase). In further embodiments, the systems may be configured for removing water dispersed in hydrocarbon fuel.

Also disclosed are methods for removing a dispersed phase (e.g., a liquid phase) from a mixture, where the mixture comprises the dispersed phase in a continuous phase (e.g., another liquid phase or a gaseous phase). The methods may include passing the mixture through the disclosed tubular surface coalescers in either on outside→in flow or an inside→out flow. In some embodiments, the methods remove at least about 80%, 85%, 90%, 91%, 92%, 93%, 94%, 95%, 96%, 97%, 98%, or 99% of the dispersed phase from a mixture of the dispersed phase in a continuous phase. In further embodiments, the methods remove water dispersed in hydrocarbon fuel.

### BRIEF DESCRIPTION OF THE DRAWINGS

FIG. 1 provides a conceptual illustration of a surface coalescence process as contemplated herein utilizing a single layer of media material.

FIG. 2 illustrates a method for determining contact angle θ for a dispersed drop on a media phase.

FIG. 3 provides a conceptual illustration of a surface coalescence process as contemplated herein utilizing two layers of media material.

FIG. 4 illustrates a setup to apply nanofibers to the outside surface of a spring via electro-spinning

FIG. 5 illustrates one embodiment of a tubular surface coalescer as contemplated herein.

FIG. 6 illustrates one embodiment of a tubular surface coalescer as contemplated herein configured for outside→inside flow.

FIG. 7 illustrates photographs of ˜3 μl water droplets on the surface of tubular surface coalescers comprising a Syndiotactic Polypropylene (SPP) nanofiber layer.

FIG. 8 illustrates a photograph of a ˜3 μl water droplet sitting on the surface of tubular surface coalescer comprising a Syndiotactic Polypropylene (SPP) nanofiber layer.

FIG. 9 illustrates a laboratory coalescer unit (i.e., a filter holder) which was used to hold modular nanofiber-coil coalescers of diameter 0.114 inch for testing and evaluation with respect to removal of water-contaminated diesel fuel.

FIG. 10 illustrates inlet water droplet size distribution (pre-filtration) for a mixture passed through the laboratory coalescer unit of FIG. 9.

FIG. 11 illustrates outlet water droplet size distribution (post-filtration) for a mixture passed through the laboratory coalescer unit of FIGS. 19 and 20.

FIG. 12 illustrates one embodiment of a tubular surface coalescer configured for inside→outside flow.

FIG. 13 illustrates one embodiment of a further configuration of a tubular surface coalescer as contemplated herein having multiple layers of media material.

FIG. 14 illustrates one embodiment of a further configuration of a tubular surface coalescer as contemplated herein having multiple types of media material.

FIG. 15 illustrates one embodiment of a further configuration of a tubular surface coalescer as contemplated herein having multiple types of media material.

FIG. 16 illustrates one embodiment of a tubular surface coalescer as contemplated herein mounted in additional media material.

FIG. 17 illustrates one embodiment of a tubular surface coalescer as contemplated herein mounted in additional media material.

FIG. 18 illustrates one embodiment of a tubular surface coalescer as contemplated herein mounted in additional media material.

FIG. 19 illustrates one embodiment of a tubular surface coalescer as contemplated herein mounted in additional media material.

FIG. 20 illustrates one embodiment of a spring for use as a porous tubular support structure for a coalescer as contemplated herein.

FIG. 21 illustrates one embodiment of a mesh or trellis tubular structure for use as a porous tubular support structure for a coalescer as contemplated herein.

FIG. 22 illustrates one embodiment of a mesh tubular structure for use as a porous tubular support structure for a coalescer as contemplated herein.

FIG. 23 illustrates one embodiment of a perforated tube for use as a porous tubular support structure for a coalescer as contemplated herein.

FIG. 24 illustrates one embodiment of an undulated tube superstructure for a coalescer as contemplated herein.

FIG. 25 illustrates one embodiment of a coiled tube superstructure for a coalescer as contemplated herein.

### DETAILED DESCRIPTION

Disclosed are coalescer elements, media material, systems and methods for coalescing a mixture of two phases, namely a continuous phase and a dispersed phase. The disclosed coalescers, systems and methods may be utilized to collect and remove the dispersed phase from the mixture. The disclosed coalescer elements may include surface coalescer elements. Particularly disclosed are tubular surface coalescers which in some embodiments may be referred to as “nanofiber-coil coalescers” or “nanofiber-coil units” and may be further described as follows.

The disclosed tubular surface coalescers, systems and methods include or utilize coalescing media material that comprises or consists one or more layers of media material applied on a porous tubular support structure. The porous tubular support structure may have a selected diameter (e.g., a diameter of about 1 mm to about 10 mm).

Suitable support structures may include spring structures such as compression springs having a distance between coils of about 0.5 mm to about 2.5 mm. Other suitable support structures include tubular mesh material, structures formed from lateral ribs supported by peripheral concentric rings, perforated hollow tube structures, and foam tube structures (e.g., a polyurethane or polyether foam tube structures which optionally may be hollow), all of which may be formed, for example, from polymeric or ceramic material. The porous tubular support structure may be made of any suitable material including metal material and polymer material.

The support structure and tubular surface coalescer formed therefrom may be flexible or bendable, and optionally may be elastic. For example, the support structure and tubular surface coalescer formed therefrom may be flexible or bent to a 90° angle, a 180° angle, or a 360° angle. The support structure and tubular surface coalescer formed therefrom may be configured into a superstructure, which may include, but is not limited to a coiled tube or an undulating tube structure.

“Flexibility,” “bendability,” and “elasticity” may be assessed as known in the art. For example, modulus of elasticity, Young's modulus, and/or Poisson's Ratio may be calculated and utilized to assess flexibility, bendability, and elasticity of a support structure or a tubular surface coalescer formed therefrom. In some embodiments, a support structure or a tubular surface coalescer formed thereofrom may have a modulus of elasticity that does not exceed 5, 4, 3, 2, 1, or 0.5 GPa.

The disclosed tubular surface coalescer may be utilized in coalescing systems. As such, the disclosed tubular surface coalescers may be modular. In some embodiments, two or more tubular surface coalescers are aligned in parallel in a coalescing system. In other embodiments, the tubular surface coalescer may be configured into a superstructure for use in the coalescing system, for example, a coiled tube or an undulating tube as described herein.

A coalescing system comprising one or more tubular surface coalescers may have a selected flow rate which may be modulated. For example, the flow rate of a coalescing system comprising one or more tubular surface coalescers may be modulated by the number of tubular surface coalescers present in the system. In addition, the flow rate of a coalescing system comprising one or more tubular surface coalescers may be modulated by the length of the one or more tubular surface coalescers.

The disclosed tubular surface coalescers, coalescing media material, systems, and methods are configured for capturing droplets of a dispersed phase from a mixture of the dispersed phase and a continuous phase. In the tubular surface coalescers, systems, and methods, the mixture is passed from upstream to downstream through one or more layers of coalescing media. As the mixture is passed through the media, droplets of the dispersed phase coalesce and grow on the upstream surface of the media to a sufficient size where they are released, drained, or collected. For example, in some embodiments, a mixture of a dispersed phase and a continuous phase may be passed through the outside surface of the one or more layers of media material applied to a porous tubular support structure, where the mixture exits through the center of the tubular structure and droplets of the dispersed phase may coalesce on the upstream surface of the media. The exiting mixture therefore comprises the continuous phase having a reduced amount of dispersed phase.

The disclosed tubular surface coalescers, systems, and methods may be utilized to coalesce any suitable mixture that includes a continuous phase and a dispersed phase. In some embodiments, the continuous phase and the dispersed phase are immiscible liquids. For example, the disclosed systems and methods may be configured or utilized for coalescing an aqueous solution (e.g., comprising mainly water) dispersed in a hydrocarbon liquid (e.g., hydrocarbon fuel, diesel fuel, biodiesel fuel, lubricating oil, hydraulic oil, or transmission oil). In other embodiments, the continuous phase is an aqueous solution (e.g., comprising mainly water) and the dispersed phase is hydrocarbon liquid. In further embodiments, the continuous phase is a gas and the dispersed phase is a liquid (e.g., hydrocarbon liquid mist). As contemplated herein, a hydrocarbon liquid primarily includes hydrocarbon material but further may include non-hydrocarbon material (e.g., up to about 1%, 5%, 10%, or 20% non-hydrocarbon material).

The coalescing media material may include at least one layer of media material that is relatively non-wettable by the dispersed phase in the continuous phase in order to facilitate coalescence of the dispersed phase at the upstream face of the media material. Optionally, the coalescing media material may include additional layers of media material that are non-wettable or wettable by the dispersed phase in the continuous phase. In some embodiments, the upstream surface (or face) of the media material is relatively smooth (e.g., by subjecting the surface to calendaring) to facilitate draining of coalesced drops of the dispersed phase.

The coalescing media described herein may comprise material having distinct hydrophilicity or hydrophobicity, or distinct oleophilicity or oleophobicity. In some embodiments, the coalescing media comprises at least one layer comprising relatively hydrophobic material, relative to the dispersed phase of the mixture of the dispersed phase in the continuous phase.

As contemplated herein, the one or more layers of coalescing media material may have a desirable pore size, capillary pressure, porosity, and solidity. The terms “pore size,” “capillary pressure,” “porosity,” “fiber diameter,” and “solidity,” may refer to “average” or “mean” values for these terms (e.g., where the layer is non-homogenous or graded and “pore size,” “capillary pressure,” “porosity,” “fiber diameter,” and “solidity,” are reported as mean pore size, average capillary pressure, average porosity, average fiber diameter, or average solidity for the non-homogenous or graded layer). These terms are further described and defined in U.S. Published Application Nos. 2011/0124,941, and 2011/0233152, the contents of which are incorporated herein by reference in their entireties.

The one or more layers of media material may have a selected mean pore size M, which may be determined by porometer. Typically, the mean pore size for at least one layer of the media material is less than the average droplet size for the dispersed phase of the mixture. The mean pore size of the layer may have a selected size. In some embodiments, 0.2 μm≦M≦12.0 μm, 2.0 μm≦M≦10.0 μm, or 4.0 μm≦M≦8.0 μm. The layer further may have a maximum pore size M_{M}. In some embodiments, the layer has a maximum pore size M_{M} and 1≦M_{M}/M≦3, 1≦M_{M}/M≦2, 1≦M_{M}/M≦1.5, or 1≦M_{M}/M≦1.25.

Typically, at least one layer of the media material is relatively non-wettable by the dispersed phase in the continuous phase. In some embodiments, the contact angle for a drop of dispersed phase in the continuous phase on the media material, 0, is no less than 90°, and in some embodiments no less than 120° (and in some embodiments no less than 135°).

In some embodiments, the media material includes at least one layer of relatively fine fibers having a mean diameter between 0.07 μm and 3.0 μm (in some embodiments between 0.15 μm and 1.5 μm) which is optionally supported on a substrate of relatively coarser fibers with a mean diameter greater than the mean diameter of the relatively fine fibers (e.g., where the relatively coarser fibers have a mean diameter greater than about 10 μm, in some embodiments greater than about 20 μm). In other embodiments, the media material includes at least one layer of a heterogenous mixture comprising relative fine fibers having a diameter between 0.07 μm and 3.0 μm (in some embodiments between 0.15 μm and 1.5 μm) and relatively coarser fibers with diameter greater than the mean diameter of the relatively fine fibers (e.g., where the relatively coarser fibers have a mean diameter greater than about 10 μm, in some embodiments greater than about 20 μm).

In some embodiments, the coalescing media material includes at least one layer having a thickness suitable for coalescing a dispersed phase in a continuous phase. In some embodiments, the coalescing media material includes at least one layer having a thickness as measured from upstream to downstream relative to flow through the layer of between about 0.05 and 0.4 mm (and in some embodiments 0.1 and 0.3 mm).

In further embodiments, the disclosed coalescing media includes at least two adjacent layers that extend in series from upstream to downstream, namely at least a first layer and at least a second layer, where the second layer may have the characteristics of a layer as described above. The first layer may have a mean pore size M₁ that is greater than the mean pore size of the second layer M₂, for example as determined by porometer. In some embodiments, M₁ is at least about 2.5 times greater than M₂ (in some embodiments at least about 5 times greater than M₂, at least about 10 times greater than M₂, or at least about 20 times greater than M₂). The mean pore sizes of the first layer and the second layer may have a selected size. In some embodiments, M₁ may be no less than about 30 μm, or in further embodiments, no less than about 180 μm). In other embodiments, 0.2 μm≦M₂≦12.0 μm, 2.0 μm≦M₂≦10.0 μm, or 4.0 μm≦M₂≦8.0 μm.

In the disclosed coalescing media comprising at least two layers, the first layer and the second layer further may have maximum pore sizes M_{M1} and M_{M2}, respectively. In some embodiments, the second layer has a maximum pore size M_{M2} and 1≦M_{M2}/M₂≦3, or 1≦M_{M2}/M₂≦2.

In the disclosed coalescing media comprising at least two layers, the first layer may include media material that is relatively wettable by the dispersed phase in the continuous phase in comparison to the second layer, and in contrast, the second layer may include media material that is relatively non-wettable by the dispersed phase in the continuous phase in comparison to the first layer. In some embodiments, the contact angle for a drop of dispersed phase in the continuous phase on layer one, θ₁, is no more than 90°, and in some embodiments no more than 45°. In further embodiments, the contact angle for a drop of dispersed phase in the continuous phase on layer two, θ₂, is no less than 90°, 120°, or 135°.

In the disclosed coalescing media comprising at least two layers, the adjacent surfaces of the first layer and the second layer (i.e., the downstream surface of the first layer and the upstream face of the second layer) may be configured to facilitate draining of coalesced drops of the dispersed phase. For example, in one embodiment of the coalescing media disclosed herein the downstream surface of the first layer may comprise fibers that are oriented in a substantially vertical direction and/or the upstream surface of the second layer may be relatively smooth (e.g., by subjecting the surface to calendaring) in order to facilitate draining of coalesced drops of the dispersed phase.

The disclosed tubular surface coalescers, systems, and methods, optionally may include or utilize a housing. The housing may include an upstream inlet structured to receive a mixture of a continuous phase and a dispersed phase, a first downstream outlet structured to discharge the cleaned mixture (with reduced dispersed phase concentration) after coalescing, and optionally a second outlet structure to discharge the coalesced dispersed phase. In some embodiments, the second outlet is on the upstream side of the media material, but downstream of the upstream inlet. The disclosed tubular surface coalescers may be mounted in the housing with their ends sealed, such that flow, either outside→inside or inside→outside, is only permitted through the tubular surface coalescers.

The disclosed tubular surface coalescers, coalescing systems, and coalescing methods typically include or utilize a single layer of media material (or optionally multiple layers of media material) for coalescing a dispersed phase from a mixture of the dispersed phase in a continuous phase. Optionally, the disclosed tubular surface coalescers, coalescing systems, and coalescing methods may include or utilize additional media (e.g., additional media positioned downstream of the coalescing media material). In some embodiments, the disclosed tubular surface coalescers, coalescing systems, and coalescing methods further may include or further may utilize an additional hydrophobic media material for removing water, where the additional hydrophobic media material is positioned downstream of the single layer of media material (or optional multiple layers of media material). In some embodiments, the disclosed tubular surface coalescers, coalescing systems, and coalescing methods further may include or further may utilize an additional media sub-layer downstream of the coalescing layer to provide structural support.

The disclosed tubular surface coalescers, systems, or methods may be utilized for removing a dispersed phase from a mixture comprising the dispersed phase in a continuous phase. In some embodiments, the disclosed tubular surface coalescers, systems, or methods may be utilized for removing water dispersed in a hydrophobic liquid, including, but not limited to, hydrocarbon fuel, diesel fuel, biodiesel fuel, lubricating oil, hydraulic oil, or transmission oil. In other embodiments, the disclosed tubular surface coalescers, systems, or methods may be utilized for removing hydrocarbon liquid dispersed in water. In further embodiment, the disclosed tubular surface coalescers, systems, or methods may be utilized to remove liquid (e.g., hydrocarbon liquid) dispersed in a gas phase. In some embodiments, the disclosed tubular surface coalescers, systems, or methods remove at least about 80%, 85%, 90%, 91%, 92%, 93%, 94%, 95%, 96%, 97%, 98%, or 99% of the dispersed phase from the mixture of the dispersed phase in the continuous phase.

The disclosed tubular surface coalescers, systems, and methods may be particularly suitable for coalescing a dispersed phase from an emulsion mixture having an interfacial tension lower than about 20 dyne/cm, or in other embodiments, lower than about 15, 10, or 5 dyne/cm. For example, the disclosed tubular surface coalescers, systems, and methods may be utilized to coalesce water from hydrocarbon liquids comprising surfactants and having an interfacial tension lower than about 20 dyne/cm, or in some embodiments, lower than about 15, 10, or 5 dyne/cm.

At least one layer of the coalescing media material utilized in the disclosed coalescers, systems, and methods typically is relatively non-wettable by the dispersed phase in the continuous phase in order to facilitate coalescence of the dispersed phase at the upstream face of the media material. In some embodiments, the media material remains relatively non-wettable by the dispersed phase in the continuous phase over long periods of exposure to the mixture, in particular, where the mixture comprises a surfactant. The coalescing media, as described herein, may comprise a layer of fibrous material (e.g., polymer, glass, ceramic, carbon, or metal fibers). In some embodiments, the coalescing media may comprise a layer of a polyester material (e.g., polybutylene terephthalate (PBT)), a polyamide material, a halocarbon material (e.g., Halar® brand ethylene chlorotrifluoroethylene (ECTFE)), or a media obtained by treating a fibrous material with an agent comprising fluorine functionalities. In some embodiments, the media may comprise PBT with 1-10% (w/w) of a fluorocarbon additive (e.g., hexafluoropropylene, hexafluoroisopropanol, hexafluoroisobutylene, and perfluorodecyl acrylate), a polyester material (e.g., PBT) compounded with 10-40% (w/w) of a fluorocarbon polymer (e.g., ECTFE), or 100% meltblown/fiber grade fluoropolymer (e.g., ECTFE).

The disclosed coalescers, systems, and methods include one or more layers of coalescing media material. In some embodiments, the disclosed coalescers, systems, and methods include or utilize a separate separator or stripping stage that is placed downstream and separated from the one or more layers of coalescing media material (e.g., hydrophobic material for removing water located downstream and separate from the single layer of media material).

The disclosed tubular coalescer elements, systems, and methods contemplated herein may include components known in the art or be utilized in systems and methods know in the art. (See, e.g., U.S. Pat. Nos. 7,416,657; 7,326,266; 7,297,279; 7,235,177; 7,198,718; 6,907,997; 6,811,693; 6,740,358; 6,730,236; 6,605,224; 6,517,615; 6,422,396; 6,419,721; 6,332,987; 6,302,932; 6,149,408; 6,083,380; 6,056,128; 5,874,008; 5,861,087; 5,800,597; 5,762,810; 5,750,024; 5,656,173; 5,643,431; 5,616,244; 5,575,896; 5,565,078; 5,500,132; 5,480,547; 5,480,547; 5,468,385; 5,454,945; 5,454,937; 5,439,588; 5,417,848; 5,401,404; 5,242,604; 5,174,907; 5,156,745; 5,112,498; 5,080,802; 5,068,035; 5,037,454; 5,006,260; 4,888,117; 4,790,947; 4,759,782; 4,643,834; 4,640,781; 4,304,671; 4,251,369; 4,213,863; 4,199,447; 4,083,778; 4,078,965; 4,052,316; 4,039,441; 3,960,719; 3,951,814; and U.S. published Application Nos. 2007-0289915; 2007-0107399; 2007-0062887; 2007-0062886; and 2007-0039865; the contents of which are incorporated herein by reference in their entireties.)

The disclosed tubular surface coalescers may be manufactured utilizing methods known in the art. (See, e.g., U.S. Pat. Nos. 8,114,183; and 7,674,425; and U.S. Published Patent Application Nos. 2007/0062887, 2007/0131235, 2011/0124,941, and 2011/0233152; the contents of which are incorporated herein by reference). For example, in some embodiments the disclosed tubular surface coalescers may be manufactured by utilizing a porous tubular structure to serve as a collector or mandrel for collecting a coalescing media material as one or more layers onto the tubular structure. In some embodiments, the coalescing material is collected on the porous tubular structure in a process that includes, but is not limited to, electro-spinning, melt-spinning, or melt-blowing. As such, the porous tubular structure functions as a collector or mandrel during manufacturing and as a support structure for the one or more layers of coalescing media material during a coalescing process. The disclosed tubular surface coalescers also may be manufactured utilizing other methods known in the art and may include additional features disclosed in the art. (See, e.g., U.S. Pat. Nos. 6,767,459; 5,443,724; and 4,081,373; and U.S Published Patent Application Nos. 2007-0131235; 2007-0062887; and 2006-0242933; the contents of which are incorporated herein by reference in their entireties).

The disclosed tubular surface coalescers may be utilized in a coalescing process. As disclosed herein, the coalescence process may be understood to comprise a series of steps including, but not limited to: (1) capture of droplets by the coalescence media material; (2) coalescence and drop growth at the upstream face of the media material; (3) drainage of coalesced drops at the upstream face of the media material; and (4) release of coalesced drops from the media material. When the coalesced drops become large enough, drag or gravitational forces induce them to flow either up or down the upstream face of the media material depending on the relative density difference of the dispersed and continuous phase. The increased droplet concentration at the upstream face of the coalescence media material and the relatively non-wetting nature of the media material facilitates the coalescence of droplets at the upstream surface of the media material. The drainage of coalesced drops from the media material may be facilitated by utilizing a media material having an upstream face with a relatively smooth surface

This invention can be applied to any set of immiscible fluids, such as water in diesel fuel, water in biodiesel fuel, oil in water, and crankcase oil from engine blow-by gases. In further embodiments, the coalescing media is present in a coalescing system that further includes a device for removing drops that are coalesced by the coalescing media. For example, a coalescing system further may include one or more of a gravity separator, centrifuge, impactor, lamella separator, inclined stacked plate, screen, quiescent chamber, and the like.

The coalescers, systems, and methods disclosed herein may include or utilize a single layer of media material, or optionally multiple layers of media material, in which coalescence mechanisms having been optimized (i.e., coalescers, systems, and methods in which the physical, structural, and surface properties of the media material have been optimized). Exemplary rules and optimal relationships among variables such as P (capillary pressure), contact angle (θ), mean pore size (M), interfacial tension (γ), porosity (ε), or solidity (1-ε) for a layer of media material may be determined as defined in the art. (See, e.g., U.S. Published Application Nos. 2011/0124,941, and 2011/0233152, the contents of which are incorporated herein by reference in their entireties.

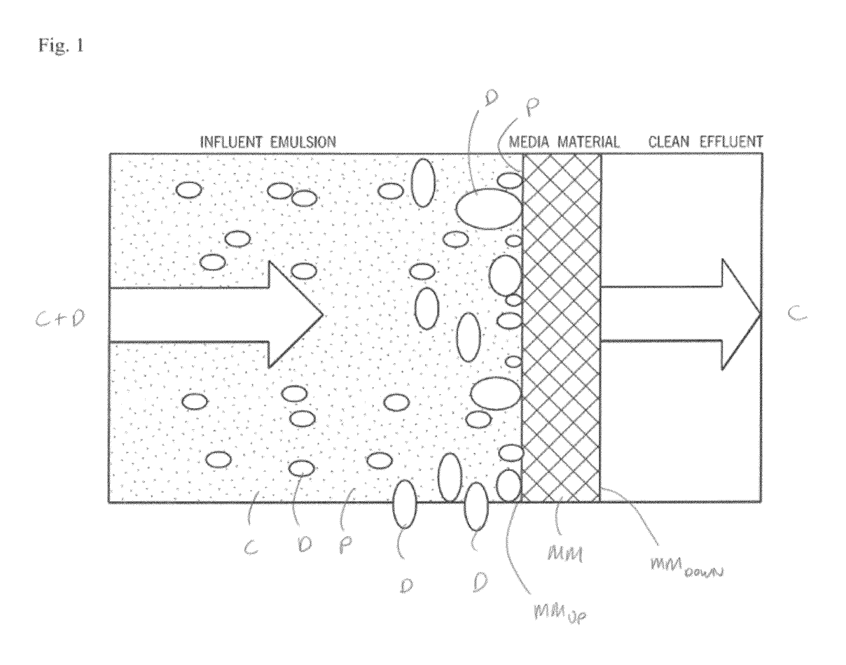

One embodiment of a surface coalescer system 10 is illustrated in FIG. 1, which performs as follows:
1. Contaminated fluid C+D consisting of droplets D (dispersed phase) suspended in a second immiscible fluid C (continuous phase), which may or may not also contain solid particulates P, flows through the system and contacts a layer of media material MM.
2. Droplets D and solid particulates P (if present) are retained on or near the upstream surface MM_{UP} of the media material MM, which acts as a barrier that prevents them from flowing through and concentrates the droplets D.
3. Filtered, cleaned continuous phase C exits the layer of media material MM, as at the downstream side MM_{DOWM}.
4. As the local concentration of captured droplets D on the upstream face MM_{UP} of the media material MM increases, they coalesce and grow which is facilitated by the relatively non-wetting character of the media material MM.
5. Coalesced drops from the upstream face of the media material MM_{UP} are repelled by the relatively non-wetting surface and drain down the face of the non-wetting upstream face MM_{UP} of the media material MM.
6. Drainage of the coalesced and wicked dispersed phase also rinses some of the capture solid particulates P from the media material MM.

The media material may be described as having at least three basic functions:
1. to prevent droplets (and solid particles) larger than a certain size from passing through,
2. to facilitate coalescence by concentrating the retained droplets on its upstream surface, and
3. to facilitate release of drops and droplets from the surface.

In some embodiments, in order to facilitate release, whether by gravity settling, drag forces, or other means, and to prevent drops from penetrating the media, the media material is highly non-wetting with respect to the dispersed phase of the mixture. In further embodiments, dispersed drops should not penetrate nor pass through the media material.

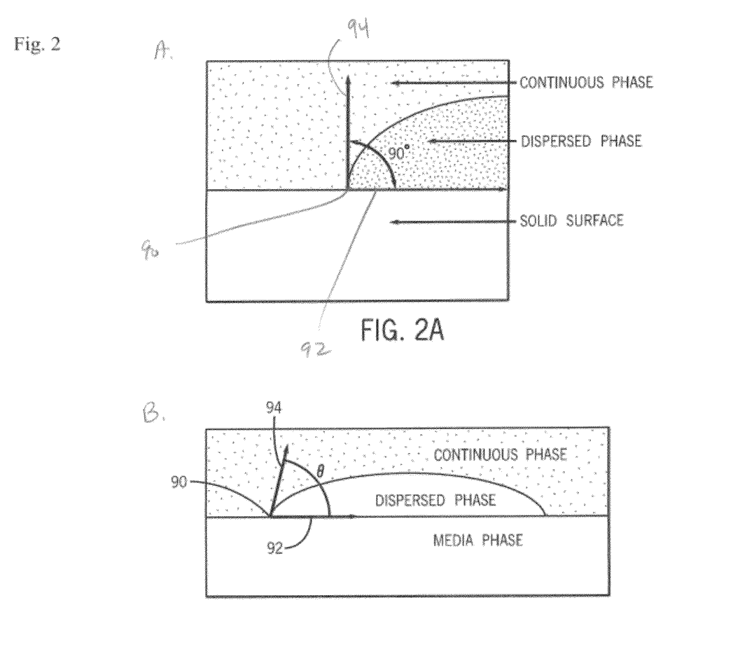

The contact angle θ of a drop of dispersed phase in continuous phase on the surface of a tubular surface coalescer element as disclosed herein may be defined as in FIGS. 2A and 2B, where θ is defined as an angle having its vertex 90 at the intersection of the continuous phase, dispersed phase, and media phase with one ray 92 extending parallel to the media surface from the vertex 90 and the other ray 94 extending tangentially to the surface of the dispersed phase at the vertex 90. In FIG. 2A, the angle θ is 90°, and is somewhat less than 90° in FIG. 2B. Typically, the angle θ is reported as being measured through the dispersed phase. In some embodiments, the contact angle may be measured for a droplet on an individual fiber of media material. In other embodiments, the contact angle may be measured for a droplet on a patch of media material. Other methods of estimating and measuring θ are known in the art.

In the media material, droplet capture typically occurs via one or more filtration mechanisms, such as diffusion, interception, inertial impaction, or sieving. For high efficiency removal of drop sizes approaching 1 μm or smaller, diffusion, interception, or sieving may be most effective. Since it is desirable for coalescence to occur on the surface of the media, as opposed to within the depth of the media as in traditional coalescers, the media of this invention is optimized to enhance removal by sieving. For the presently disclosed coalescing media, the pore size of the media material, M, typically is smaller than the smallest drop size that is desired to be removed.

For fuel water removal in high pressure common rail systems (HPCR), the system is designed to remove drops smaller than ˜3 μm. Drops smaller than ˜0.2 μm can, in some embodiments, be considered to be dissolved or reverse micelles. In some embodiments, the media material may comprise a thin layer of fibers with diameter between 0.07 μm and 3.0 μm, the thin layer of fibers being formed of overlapping and intersecting fibers that provide a pore size between 0.2 and 12 μm, (or in some embodiments between 2.0 and 10 μm, or between 4.0 and 8.0 μm). The thin layer of fibers optionally is supported on a substrate of coarser fibers having a mean diameter greater than the mean diameter of the fine fibers (e.g., where the relatively coarser fibers have a mean diameter greater than about 10 μm or greater than about 20 μm). In further embodiments, the media material may comprise a heterogenous layer of mixed fibers comprising fine fibers with a mean diameter between 0.07 μm and 3.0 μm and coarser fibers having a mean diameter greater than the mean diameter of the fine fibers (e.g., where the relatively coarser fibers have a mean diameter greater than about 10 μm, and in some embodiments greater than about 20 μm). In some embodiments, the heterogenous may have a mean pore size between 0.2 and 12 μm, (or in further embodiments between 2.0 and 10 μm, or between 4.0 and 8.0 μm).

In some embodiments, in order to achieve high efficiency, low pressure drop, or increased life, it may be desirable to use fibrous, non-woven media material, as opposed to membrane or granular material. Fine fibers between 0.07 and 3.0 μm an typically have the capability to yield both low pressure drop and high efficiency. However, when used alone, these fibers may lack the structural characteristics to prevent collapse or burst. Thus, in some embodiments, a combination of fine fibers and coarser structural fibers may be utilized. This can be accomplished in several ways, including, but not limited to, using:
1. A layer of fine fibers bonded to or supported by a substrate of structural fibers, e.g., meltblown polyester, other polymeric fibers, microglass, cellulose or other suitable structural fibers. This may be achieved by electrospinning or otherwise producing and laying down a nanofiber layer onto a substrate of filter media composed of coarser fibers, such as 3 to 30 μm polyester fibers. The nanofiber layer typically has the capability to yield both low pressure drop and high efficiency. The structural fibers provide support, and may allow for pleating and processing. The two layers may be attached to one another through ultrasonic bonding, the use of adhesives, physical constraints, or simply by allowing the freshly produced, warm, tacky, unsolidified nanofibers to cool and adhere to the support fibers.
2. A parent filter media composed primarily of coarser structural fibers greater than 1 μm, 10 μm, or 20 μm, e.g., meltblown polyester FWS media, microglass, and impregnated with carbon nanotubules smaller than 0.3 μm in diameter. The substrate material may be polymeric, e.g., polyester, nylon, polypropylene, polyphenylene sulfide, polyurethane, fluorocarbon, a thermoplastic polymer, or other polymeric material that can be formed into a non-woven fibrous or other porous structure. The substrate may be formed into a non-woven fibrous structure by wet laying, melt blowing, melt spinning, or other suitable process. The substrate media is then processed such that carbon nanotubules are incorporated into the media to bridge the micropores formed by the coarse fibers with carbon nanotubule nanofibers, such as described in U.S. Pat. Nos. 7,211,320 and 7,419,601 (which are incorporated herein by reference in their entireties); by thermally bonding the nanotubules to the parent media; or through the use of resin or other binders to attach the nanotubules to the parent media.

Non-woven filter media typically comprises pores and fibers of a range of different sizes. For high removal efficiency coalescence (e.g., greater than ˜98%), the range of M may be controlled. As discussed above, M is the mean pore size of the media material. Mean pore size may be determined by a porometer. For high efficiency coalescers, the maximum pore size, M_{M}, may be controlled. Specifically, the ratio of M_{M} to M, the pore size ratio, may meet the criteria 1≦M_{M}/M≦3.

In some embodiments, this ratio is controlled in the design of high efficiency surface coalescers, since the flow of the emulsion will tend to preferentially pass through larger, more open pores, as opposed to smaller, more restrictive pores in the media. Thus, high removal efficiency preferably includes controlling the maximum pore size, where it is preferably that the maximum pore size is close to the mean pore size. As a previously unrecognized secondary benefit, a media that has a pore size ratio that is close to 1 will have a narrower pore size distribution and a more uniform surface that is easier for drops to drain from. Therefore, in preferred embodiment, the pore size ratio for the media material is less than 3, more preferably less than 2, and even more preferably approaches 1. Thus, in some embodiments, 1≦M_{M}/M≦3, in other embodiments, 1≦M_{M}/M≦2, in other embodiments, 1≦M_{M}/M≦1.5, and in other embodiments, 1≦M_{M}/M≦1.25.

In addition to these physical characteristics, the contact angle of a drop of the dispersed phase in the continuous phase on the media may be an important characteristic. In a some embodiments, the discloser tubular coalescers include at least one layer of media material that is relatively non-wetted by the dispersed phase in the continuous phase (e.g., where θ≧120°, and, ideally, θ≧135° in order to retain drops for surface coalescence and to prevent passage of droplets through the media material. A highly non-wetting θ may be obtained in a number of commercially available ways to achieve non-wetting properties of the surface of the media material. For fuel water separators and other applications where water is the dispersed phase and a hydrocarbon liquid is the continuous phase (e.g., lube or hydraulic oil), the media material typically is hydrophobic and methods for obtaining a hydrophobic media material include, but are not limited to:
1. use of polymeric fibers with inherently hydrophobic properties, such as fluorocarbon fiber (e.g., Halar®ECTFE (a copolymer of ethylene and chlorotrifluoroethylene), polytetrafluoroethylene, or other fluorocarbon polymer), polyester (e.g., polybutylene terphthalate or other hydrophobic polyester), polypropylene, polyethylene, polyphenylene sulfide, polysulfone, acetal, and the like.
2. treatment of a base polymer, glass, metal, ceramic, or carbon fiber media with fluorocarbon or silicone resins, or surfactants (e.g., Rain-X® brand glass treatment) to impart hydrophobicity
3. plasma treatment of the media with a plasma containing fluorine substituents such as are described in U.S. patent application Ser. No. 12/247,502 and in Plasma Surface Modification and Plasma Polymerization, N. Inagaki, CRC Press, NY, 1996, which contents are incorporated herein by reference in their entireties.

For crankcase ventilation, similar methods may be used. However, the resultant surface typically is oleophobic. For example, the surface may include fluorocarbon functionalities. In applications of the coalescing media for removing oil or non-polar droplets from water, coolants, or other polar fluids, an oleophobic or hydrophilic surface may be obtained by methods that include, but are not limited to:
1. use of mineral oxide (e.g., glass, silica, ceramic), metal or polymeric fibers with inherently hydrophillic properties, such as nylon 6,6 or other hydrophilic polyamides, glass or ceramic, hydrophilic polyurethanes, polyvinyl alcohols, other hydrophilic polymers or oleophobic fluorocarbon media.
2. plasma treatment of the media with a plasma containing fluorine, oxygen, or nitrogen substituents, such as described in Plasma Surface Modification and Plasma Polymerization, N. Inagaki, CRC Press, NY, 1996, which content is incorporated herein by reference in its entirety.

In some embodiments, the orientation of the disclosed coalescing media in a coalescer is important for optimal function. For example, in some embodiments drainage may be vertically downward in the direction of gravity where the dispersed phase has a relative density that is greater than the relative density of the continuous phase. In other embodiments, where the dispersed phase has a relative density that is lower than the relative density of the continuous phase, the dispersed phase may collect as drops which subsequently flow upward.

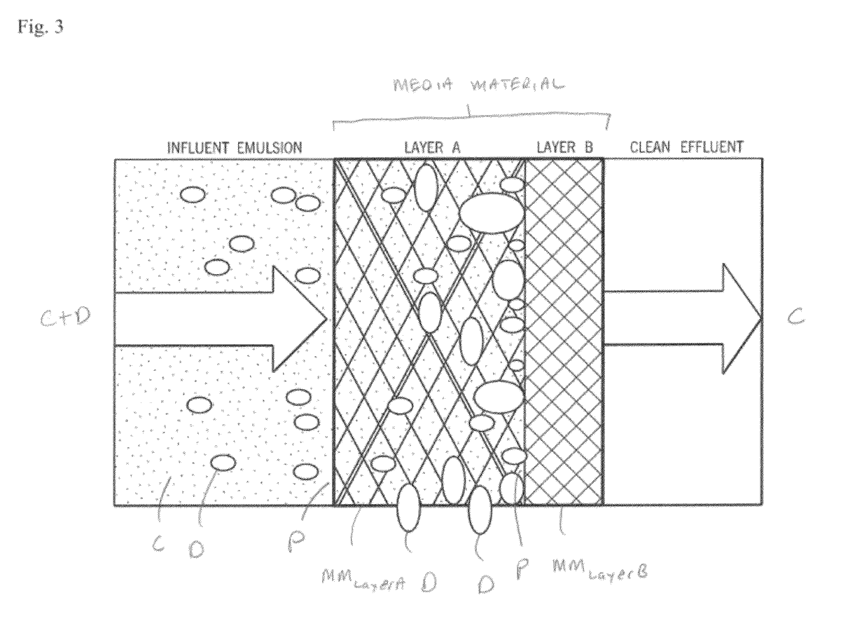

In some embodiments, the disclosed surface coalescing media comprises or consists of a single layer of coalescing media material. In other embodiments, the disclosed coalescing media includes upstream drainage/prefilter layer (e.g., “Layer A”) in addition to a layer of coalescing media material (e.g., “Layer B”). One embodiment of a surface coalescer 100 is illustrated in FIG. 3, which performs as follows:
1. Contaminated fluid C+D consisting of droplets D (dispersed phase) suspended in a second immiscible fluid C (continuous phase), which may or may not also contain solid particulates P flow through the first layer of media material MM_{LayerA}.
2. In the first layer of media material MM_{LayerA} some of the droplets D and solid particulates P, primarily the larger ones, are captured and retained.
3. Droplets D and solid particulates P not captured by Layer A flow therethrough and are retained on or near the upstream surface of the second layer of media material MM_{LayerB} that acts as a barrier that prevents them from flowing through and concentrates the droplets D.
4. Filtered, cleaned continuous phase C exits Layer B as at downstream side.
5. As the local concentration of captured droplets on the surface of Layer B increases, they coalesce and grow which is facilitated by the presence of relatively wetting Layer A.
6. Coalesced drops from the surface of Layer B are repelled by the relatively non-wetting surface and optionally are wicked back into Layer A (e.g., where the contact angle of Layer A is less than about 90°), or alternatively, the coalesced drops may drain down the face of the non-wetting surface of Layer B.
7. As the dispersed phase wetting surface of Layer A becomes saturated, dispersed phase drains it under the influence of gravity, pressure or other force.
8. Drainage of the coalesced and wicked dispersed phase also rinses some of the capture solid particulates from the media.

The downstream layer (Layer B) has three basic functions similar to the single layer of media material discussed above.
1. to prevent droplets (and solid particles) larger than a certain size from passing through,
2. to facilitate coalescence by concentrating the retained droplets on its upstream surface, and
3. to facilitate release of drops and droplets from the surface.
   The downstream layer (Layer B) may share one or more characteristics of the single layer of media material as discussed above. The characteristics of the downstream layer (Layer B) may be determined or modulated based on the equations discussed above for the single layer of media material.

The optional first layer, i.e., “Layer A,” typically serves a different function than the second layer, i.e., “Layer B.” Typically, the function of Layer A is to:
1. provide a region of higher capillary pressure than Layer B and optionally to assist in wicking captured and coalesced drops and droplets away from the surface of Layer B,
2. facilitate drainage of captured and coalesced drops and droplets from the media,
3. facilitate coalescence of captured droplets, and
4. optionally, serve as a prefilter for solids or other contaminants that may prematurely plug the media.

In order to facilitate wicking and coalescence of captured droplets, the first layer preferentially is more wettable by the dispersed phase than the second layer (i.e., θ_{(Layer A)}<θ_{(Layer B)}. Drainage of the dispersed phase, including drops and droplets from this layer typically is facilitated by having a large pore size. Where it is desirable that Layer A function as a pre-filter, the pore size of Layer A may be larger than that of Layer B. In some embodiments, a multilayer or multimedia pre-filter may precede Layer A (i.e., be upstream of Layer A) in order to maximize the life and extend the service interval of the media disclosed herein. Also, in some embodiments, Layer A is wetted by the dispersed phase (e.g., where θ≦90°, θ≦60°, or θ≦45°).

For fuel-water coalescers and other applications where water is the dispersed phase and a hydrocarbon liquid is the continuous phase (e.g., lube or hydraulic oil), Layer A is relatively hydrophilic compared to Layer B. Methods to achieve relatively hydrophilic surfaces are described above.

For crankcase ventilation and for the removal of oil or non-polar droplets from water, coolants, or other polar fluids, similar methods may be utilized. However, typically Layer A is relatively oleophilic compared to Layer B and methods to achieve oleophilic surfaces are described above. In some embodiments, Layer B may comprise PBT with 1-10% (w/w) of a fluorocarbon additive, a polyester material compounded with 10-40% (w/w) of a fluorocarbon polymer, or 100% meltblown/fiber grade ECTFE

In some embodiments, the orientation of the disclosed coalescing media in a coalescer is important for optimal function. Drainage will be vertically downward in the direction of gravity. Wicking, will typically involve horizontal transport of droplets from the surface of Layer B, but other orientations are possible. Typically, the desired direction of drop transport for wicking does not oppose gravity. As long as Layer A is preferentially wetting, wicking should occur, even if M is so large that there is negligible capillary pressure. However, in some embodiments, M may be an important design consideration for drainage. In order to function over a range of γ from 5 to 15 dyn/cm, in some embodiments M>30 μm for Layer A and, in further embodiments M≧180 μm for Layer A.

In some embodiments, Layer A may comprise fibers that are substantially oriented in a vertical direction (e.g., in an axis that is parallel to gravity). For example, Layer A may comprises fibers that are substantially oriented in a vertical direction at the downstream surface or face of Layer A that is adjacent to the upstream surface or face of Layer B (FIG. 3) in order to facilitate drainage of droplets from the surface of Layer B. Media material for Layer A having fibers that are substantially oriented in a vertical direction may be prepared by subjecting a surface of the media material to a “carding” process which parallelizes the fibers of the surface. In some embodiments, media that comprises fibers that are substantially oriented in a vertical direction (e.g., fibers on a downstream surface or face) means media wherein at least about 70%, 80%, or 90% of the fibers are substantially vertical (e.g., deviating from a vertical axis by no more than 30, 20, or 10 degrees). In further embodiments, the upstream surface of Layer B may be relative smooth, for example, where the surface has been subject to a calendaring process.

The surface coalescer may comprise a single multilayer media, such as formed by melt-blowing two different layer of media, once of top of another, electro-spinning, melt-spinning, or other means or combination of means or processes. Alternatively, the surface coalescer may comprise two distinct filter media with the specified properties held in intimate physical contact by pleating, pressure, adhesives, bonding resins, ultrasonic bonding, thermal bonding or other means.

### ILLUSTRATIVE EMBODIMENTS

The following embodiments are illustrative and are not intended to limit the scope of the claimed subject matter.

Embodiment 1. A tubular surface coalescer comprising one or more layers of coalescing media material applied to an outer surface of a porous tubular support structure.

Embodiment 2. The tubular surface coalescer of embodiment 1, wherein the porous tubular support structure has a diameter of between about 1 mm and about 10 mm.

Embodiment 3. The tubular surface coalescer of any of the foregoing embodiments, wherein the porous tubular support structure is a spring.

Embodiment 4. The tubular surface coalescer of embodiment 3, wherein the spring has an average distance between coils of about 0.5 mm and about 2.5 mm.

Embodiment 5. The tubular surface coalescer of embodiment 1, wherein the porous tubular structure is a tubular mesh.

Embodiment 6. The tubular surface coalescer of embodiment 1, wherein the porous tubular structure is a perforated hollow tube.

Embodiment 7. The tubular surface coalescer of embodiment 1, wherein the porous tubular structure is a foam tube.

Embodiment 8. The tubular surface coalescer of any of the foregoing embodiments, wherein the coalescing media material comprises fibers having a mean diameter between about 0.05 μm and 5 μm.

Embodiment 9. The tubular surface coalescer of embodiment 8, wherein the fibers are polymeric fibers.

Embodiment 10. The tubular surface coalescer of embodiment 8, wherein the fibers are ceramic fibers.

Embodiment 11. The tubular surface coalescer of embodiment 8, wherein the fibers are carbon fibers.

Embodiment 12. The tubular surface coalescer of any of embodiments 8-11, wherein the fibers are applied to the outer surface of the porous tubular support structure via electro-spinning, melt-spinning, or melt-blowing.

Embodiment 13. The tubular surface coalescer of any of the foregoing embodiments, wherein the coalescing media material has a mean pore size M, wherein 0.2 μm≦M≦12.0 μm.

Embodiment 14. The tubular surface coalescer of any of the foregoing embodiments, wherein the media material has a maximum pore size M_{M} and 1≦M_{M}/M≦3.

Embodiment 15. The tubular surface coalescer of any of the foregoing embodiments, comprising at least two adjacent layers of coalescing media material applied to the outer surface of the porous tubular support structure, namely an outer first layer of media material and an inner second layer of media material, and the first layer and the second layer having mean pore sizes M₁ and M₂, respectively, and M₁>M₂.

Embodiment 16. The tubular surface coalescer of embodiment 15, wherein M₁ is at least about 2.5 times greater than M₂.

Embodiment 17. The tubular surface coalescer of embodiment 15, wherein M₁≧30 μm.

Embodiment 18. The tubular surface coalescer of embodiment 15, wherein 0.2 μm≦M₂≦12.0 μm.

Embodiment 19. The tubular surface coalescer of embodiment 15, wherein the outer first layer of media material comprises media having an average fiber diameter that is less than about 100 μm.

Embodiment 20. The tubular surface coalescer of embodiment 15, wherein the coalescing media material is formed by electro-spinning, melt-spinning, or melt-blowing the inner second layer of media material on the porous tubular support structure, and subsequently electro-spinning, melt-spinning, or melt-blowing the outer first layer of media material on the inner layer of media material.

Embodiment 21. The tubular surface coalescer of embodiment 1, wherein the coalescer is flexible.

Embodiment 22. The tubular surface coalescer of embodiment 1, configured as a coiled tube.

Embodiment 23. The tubular surface coalescer of claim 1, configured as an undulating tube.

Embodiment 24. The tubular surface coalescer of any of the foregoing embodiments contained in a housing, the housing having an upstream inlet structured to receive a mixture and a downstream outlet structured to discharge the mixture after coalescing of the dispersed phase.

Embodiment 25. A coalescing system comprising the tubular surface coalescer of any of embodiments 1-24.

Embodiment 26. The coalescing system according to embodiment 25, configured for removing water dispersed in hydrocarbon fuel.

Embodiment 27. The coalescing system of embodiment 25 or 26, wherein the system comprises one or more of the tubular surface coalescers aligned in parallel.

Embodiment 28. The coalescing system of any of embodiments 25-27, wherein the system has a flow rate determined by total number of the one or more tubular surface coalescers present in the system.

Embodiment 29. The coalescing system of any of embodiments 25-28, wherein the system has a flow rate determined by length of the one or more tubular surface coalescers present in the system.

Embodiment 30. A method of removing water dispersed in hydrocarbon fuel, the method comprising passing a mixture comprising hydrocarbon fuel and water dispersed in the hydrocarbon fuel through the tubular surface coalescer of any of embodiments 1-24 and removing at least about 90% of the water dispersed in the hydrocarbon fuel.

Embodiment 31. The method of embodiment 30, wherein the ratio of the diameter of the porous tubular support structure to the average diameter of the water drops dispersed in the hydrocarbon fuel is less than about 100.

Embodiment 32. A method of removing hydrocarbon fuel drops dispersed in water, the method comprising passing a mixture comprising water and hydrocarbon fuel dispersed in the hydrocarbon fuel through the tubular surface coalescer of any of embodiments 1-24 and removing at least about 90% of the hydrocarbon fuel dispersed in the water.

Embodiment 33. The method of embodiment 32, wherein the ratio of the diameter of the porous tubular support structure to the average diameter of the hydrocarbon drops in the water is less than about 100.

Embodiment 34. A method of removing hydrocarbon droplets dispersed in an airstream, the method comprising passing a mixture comprising an airstream and hydrocarbon mist dispersed in the airstream through the tubular surface coalescer of any of embodiments 1-24 and removing at least about 90% of the hydrocarbon mist dispersed in the airstream.

Embodiment 35. The method of embodiment 34, wherein the ratio of the diameter of the porous tubular support structure to the average diameter of the hydrocarbon droplets dispersed in the airstream is less than about 100.

### EXAMPLES

The following examples are illustrative and are not intended to limit the scope of the claimed subject matter.

In one example, a nanofiber mat structure is combined with a tubular geometry structure to make a tubular surface coalescer. The nanofibers are electro-spun directly onto the outer surface of a coiled compression spring or similar supporting tubular structure in a layer. The spring provides a support structure for the nanofiber mat structure. The spaces or gaps between the spring's coils allow the passage of the oil, but are small (short) enough to provide the structural strength needed to support the nanofibers without tearing. In this example, the nanofibers are spun directly onto a supporting structure that is a semi-rigid structural part of the tubular surface coalescer. This is in contrast to prior art, in which case, the nanofiber is either spun onto a supporting web (such as flat sheet media) that is later supported by a rigid structural part, such as a center tube or stand pipe, or onto a collector or mandrel which is part of the production equipment. The tubular support structure in this example serves the dual function of supporting the nanofiber in actual use and of supporting the nanofiber during manufacturing. By using small diameter springs as a support, the effective size of a coalescer system and its dimensions can be selected based on the number and size of tubular surface coalescers used in the coalescer system. As such, the tubular surface coalescers are modular. Further, the tubular surface coalescer may be bent or flexible so that they can be utilized in a wide variety of system configurations.

In the present example, an appropriate polymer for the nanofiber layer may be selected such that the contact angle of dispersed liquid drops exceeds 150°. For example, polypropylene nanofibers produce water contact angles in excess of 160° in air and also when submerged in diesel fuel. In the present example, the dispersed liquid phase (droplets) may have a high contact angle with the nanofiber layer, for example greater than 135°, and the continuous phase has a low contact angle with the nanofiber layer, for example less than 90°, in order for the nanofiber layer to provide good separation of the dispersed liquid phase and the continuous phase. In such configurations, the dispersed phase is rejected by the nanofiber layer while the continuous phase flows through the layer. In other examples, the opposite configuration may be utilized, in which the continuous phase is rejected by the layer (high contact angle) and the dispersed phase (low contact angle) passes through the layer.

In this example, the tubular surface coalescers have a relatively small diameter (e.g., between about 1 mm and about 10 mm). This relatively small diameter provides the tubular surface coalescers with enhanced wettability properties, which accordingly enhance the performance of the tubular surface coalescers. Drops sitting on the surface of coalescer media have an inherent contact angle, that is a function of the properties of the coalescer media, the dispersed phase (e.g., in water-in-fuel water separation applications), and the continuous phase (e.g., in fuel-in-water separation applications). The interfacial tension (force) that holds a drop to a surface is directly related to the length of the contact line between the drop and the surface. For a drop attached to outside surface of a tube, the contact line is shorter in length than that of a similar volume drop and similar interfacial tension attached to a flat surface. This is a significant advantage for the tubular geometry because it is easier for drops to separate (i.e., drip off or migrate due to gravity) from the tube surface. In order to assess differences in contact angles for drops sitting on flat surfaces versus tubular surfaces, the contact angle of water drops on nanofibers of 2% (w/w) polypropylene solution that were electro-spun in flat sheet form were compared to the contact angle of water drops on nanofibers of 2% (w/w) polypropylene solution that were electro-spun onto a spring having a 0.094 inch outer diameter and being formed of a wire having a diameter of 0.013 inch. The average contact angle of a water drop on the flat surface in air was observed to be 149.8° with a standard deviation of 0.62°, while on the curved nanofiber on spring surface, it was observed to be 158.4°5 with a deviation of 2.12°. This demonstrates how wettability is affected by the geometry of the surface. In this case, hydrophobity was enhanced by curvature of the surface, which in turn enhances coalescence. As separation proceeds, the localized surface concentration of droplets on the curved surface increases, thus enhancing coalescence of the droplets into larger drops. These larger drops are more easily drained from the small diameter hydrophobic tubes as compared to flat surfaces, (e.g., flat pleated surfaces). As the larger drops drain downward, they clear the surface of the tubular surface coalescer for subsequent drops to be captured and coalesced. Furthermore, small diameter tubes are advantageous because they are more compact and easy to handle.

The geometry of the disclosed tubular surface coalescers thus enhances the effective hydrophobicity of the surface of these coalescers which reduces transport of drops through the media and increases drainage of captured droplets from the surface of the coalescers. The magnitude of this enhancement in effective hydrophobicity achieved by utilizing a curved surface rather than a flat surface is a function of the at least three variables: (1) inherent contact angle, (2) the radius of curvature of the surface, and (3) the radius of the drop. Coalescer systems can be configured to optimize this enhancement in effective hydrophobicity applying knowledge of the relationship between these three (3) variables. In general, it should be noted that the magnitude of the effective hydrophobicity enhancement will increase as the ratio of the drop radius to the radius of curvature of the media increases. In some embodiments, the ratio of the drop radius to the radius of curvature of the media is from 3 to 1, in other embodiments, from 2 to 1, and in other embodiments, from 1.5 to 1.

The critical ratio can be estimated by comparing the maximum drop density to the ratio for a given drop radius, wherein maximum drop density equals the number of drops with a contact angle of 180° (i.e., non-wetted) per unit length that can be loaded onto the surface of the media. Such an analysis indicates that the ratio of the drop radius to the radius of curvature of the media should be less than 100, and ideally less than 10. It should also be noted that, while the example of fuel-water coalescence is used in this example, the underlying principles can be applied to other coalescers or separators used to separate two immiscible phases, such as those previously described.

In the present example, the tubular surface coalescers may be otherwise referred to as “nanofiber-coil coalescers” or “nanofiber-coil units.” The nanofiber-coil units can be made with spun nanofibers (fiber diameter <500 nm) on the support structure tubes with a range of tube diameters (<10 mm diameter). The nanofibers may be made hydrophilic and to have a contact angle less than 45° to attract small water drops less than 50 μm in diameter, allowing them to coalesce on the nanofiber surface. Ideally, the pore size should be less than 1 μm. In a further example, a hydrophilic nanofiber layer may be supported by a hydrophobic fibrous layer wrapped or spun around the support structure springs or hollow tubes. Ideally, the hydrophobic fibrous layer may be: (a) a nanofiber layer having a contact angle less than 90° with pore size less than 1 μm in diameter, (b) a hydrophobic mesh screen with pore diameter less than 1 μm, (c) or a melt-blown polyester scrim with pore diameter of less than 1 μm. In this example, the hydrophilic nanofiber layer attracts the water droplets in the fuel to its surface, where they reside until coalesce occurs and they become large enough to drain due to gravity and/or rejection by the hydrophobic composite layer on the support structure tube or spring.

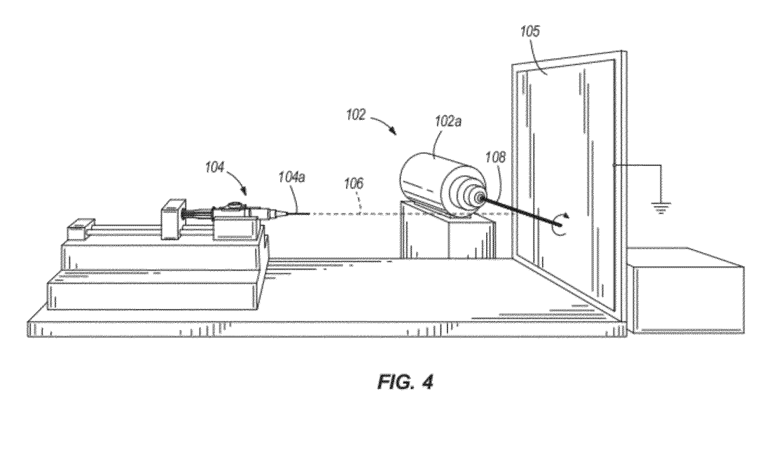

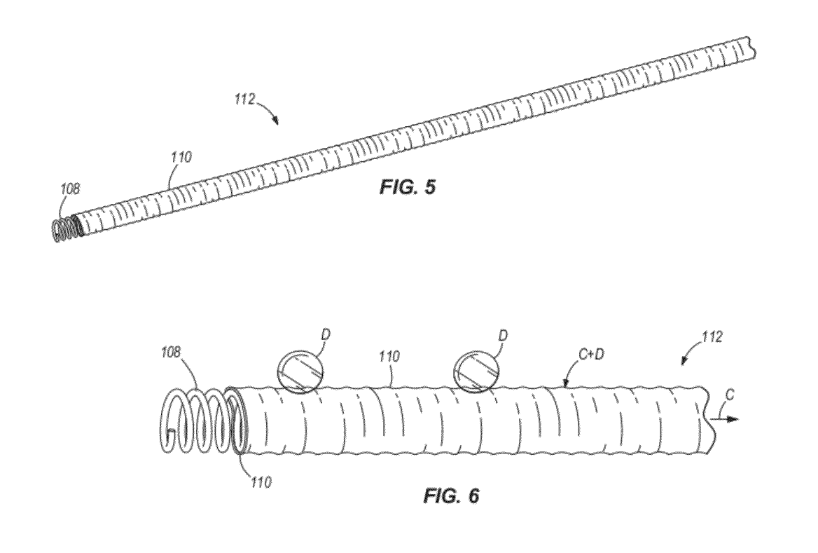

In this example, experiments were conducted on syndiotactic polypropylene to study the hydrophobic behavior, and to demonstrate water separation (i.e., rejection) while still allowing passage of diesel fuel. Initially, the hydrophobic behavior of Syndiotactic Polypropylene was studied. The Syndiotactic Polypropylene (SPP, melt index: 2.20 g/10 min at 230° C. with a load 2.16 Kg, ASTM D 1238) used was purchased from Aldrich Chemical (Milwaukee, Wis.). The weighted average (M_{W}) and number-average molecular weight (M_{N}) are 174,000 and 75,000 g/mol, respectively (M_{W}/M_{N}=2.32). SPP was dissolved in a solvent mixture of cyclohexane, acetone and dimethylformamide (DMF) (80/10/10 by weight ratio) at 70° C. at a weight concentration of SPP to solvent mixture varying from 1.5% to 3%. This polymer mixture was subjected to electro-pinning as described by Lee et al., “A review of recent results on super-hydrophobic materials based on micro- and nanofibers.” Journal of Adhesion Science and Technology (2008), 22(15), 1799-1817. Methods were developed to electro-spin a continuous uniform mat of polymer nanofibers onto coiled springs. FIG. 4 illustrates the electro-spinning apparatus. The polymer solution was loaded in the syringe 104, and the syringe needle 104a was charged to about 25 KV. The spring 108, motor 102 and background aluminum foil 105 were grounded, and the motor was covered with foil 102a to protect the motor 102 from effluent fibers 106. The spring 108 was rotated and utilized to collect effluent fibers 106 to form a coating 110. (See FIGS. 5 and 6). FIGS. 5 and 6 illustrate the appearance of a modular nanofiber-coil coalescer 112 prepared as such. Flow of a mixture of a continuous and dispersed phase C+D through the coalescer is outside→inside with the dispersed phase D coalescing on the outside of the nanofiber coating 110 and the continuous phase C exiting axially through the coalescer. After passing through the coalescer, the continuous phase C will contain a reduced amount of dispersed phase D, but still may contain a small percentage of dispersed phase D.

The conditions used for preparing the modular nanofiber-coil coalescer 112 and resultant contact angles for water on the surface of the coalescers are shown in Table 1.

TABLE 1
Electro-spinning specifications and water contact angle values

| | | Thick- | Electro-spinning Specifications | | | |
|---|---|---|---|---|---|---|
| S. | Weight | ness | Distance | Voltage | Flow rate | Contact |
| NO | % | (mm) | (cm) | (KV) | (ml/h) | Angle |
| 1 | 1.5 | 0.69 | 25 | 25 | 25 | 160.6 ± 5.03 |
| 2 | 2 | 0.49 | 25 | 25 | 25 | 163.1 ± 4.01 |
| 3 | 2.5 | 0.72 | 25 | 16 | 25 | 161.7 ± 4.28 |
| 4 | 3 | 0.52 | 10 | 10 | 20 | 146.2 ± 4.92 |

Tempered steel compression springs of outer diameter 0.094 inch (wire diameter: 0.013 inch) and 0.156 inch (wire diameter: 0.016 inch), as well as rods of similar outer diameter (OD) were utilized as support structure to prepared coalescers. The coalescers then were tested with respect to contact angle on the surface of the coalescers. The water contact angle on the spring with OD 0.156 inch was difficult to measure, because the water droplet sat between the coils of the spring because of a larger pitch of the spring.

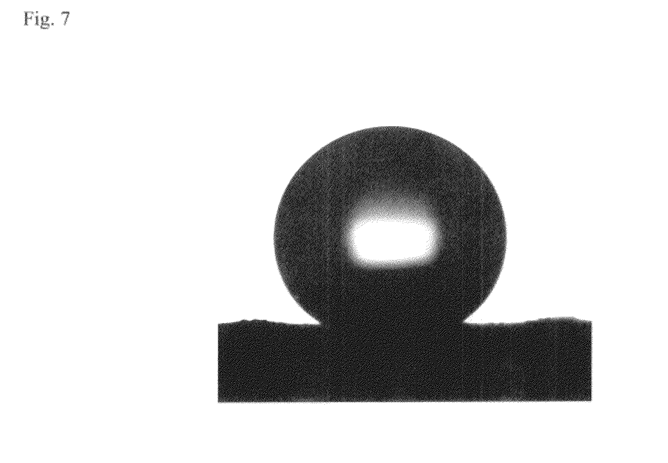

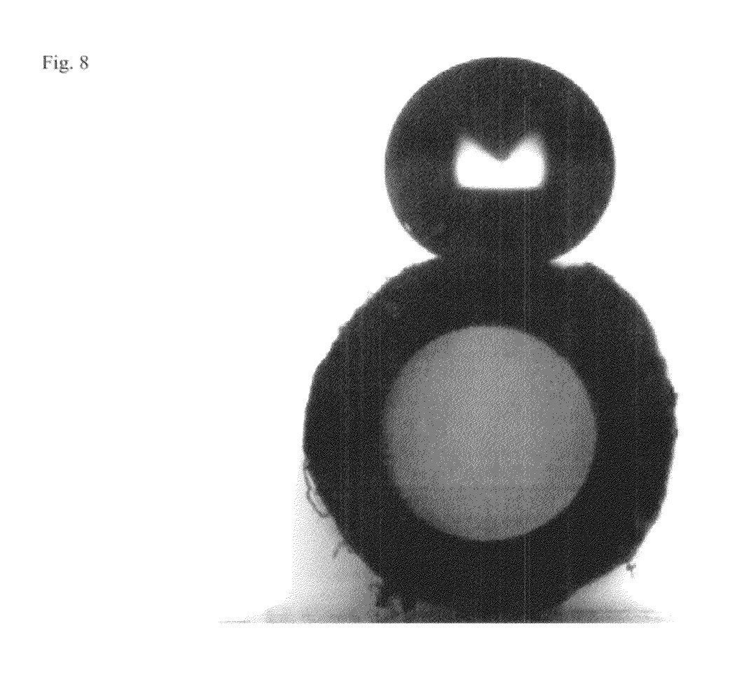

The contact angle of a drop of water on a flat surface of media was compared to the contact angle of a drop of water on a curved surface of media of a tubular coalescer. FIG. 7 and FIG. 8 illustrate the contact angle of a drop of water on the surface of flat media versus curved media. As illustrated, the drop on the curved surface (FIG. 8) sits higher (i.e., has a higher contact angle) than the drop on the flat surface (FIG. 7).

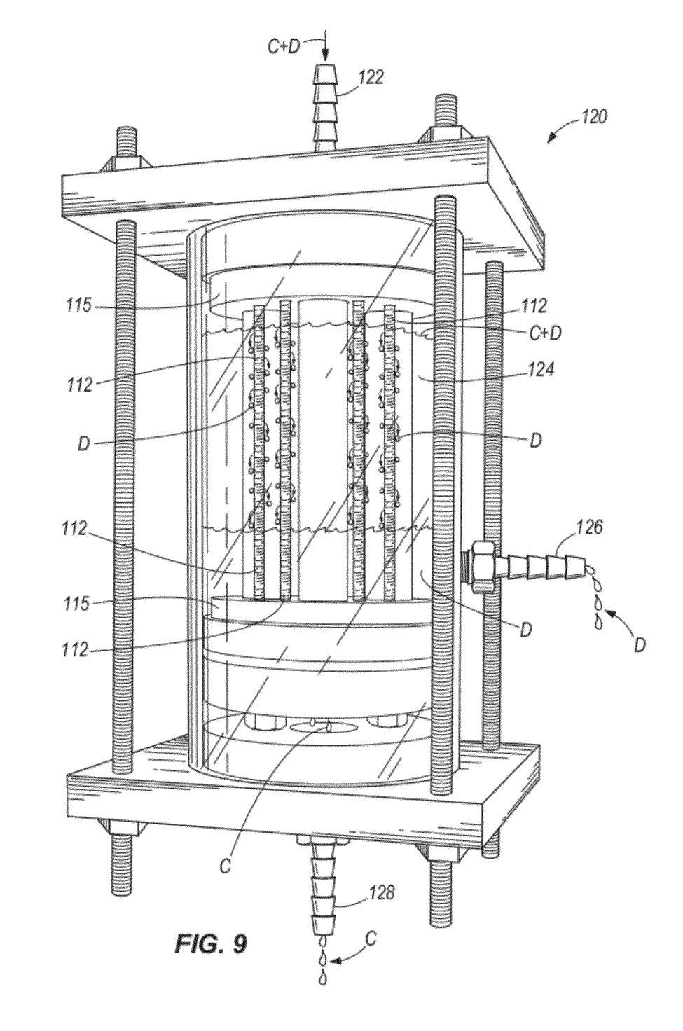

A coalescer unit 120 which contained nanofiber-coil coalescers as disclosed herein was tested as shown in FIG. 9. Nanofiber-coil coalescers 112 were mounted in the coalescer unit in polyurethane 115. The diesel fuel flow rate at the top inlet 122 was maintained at 170 ml/hr using a peristaltic pump. Water was added to the diesel fuel upstream of the filter through a needle (0.8 mm) at a flow rate of 150 μl/min to provide a model mixture of water dispersed in diesel fuel C+D which entered the unit 120 at the top inlet 122. The mixture collected in a housing 124 of the unit 120. As the mixture C+D was passed through the coalescers 112 in an outside→inside direction the water dispersed in the diesel fuel coalesced as drops D on the outside of the coalescers 112. The coalesced drops D drained downward and collected at the bottom of the housing 124. The collected water then exited the unit 120 at a side outlet 126. The filtered mixture which mainly is comprised of diesel fuel C and has a reduced amount of water content D, exits the unit 120 at a bottom outlet 128.

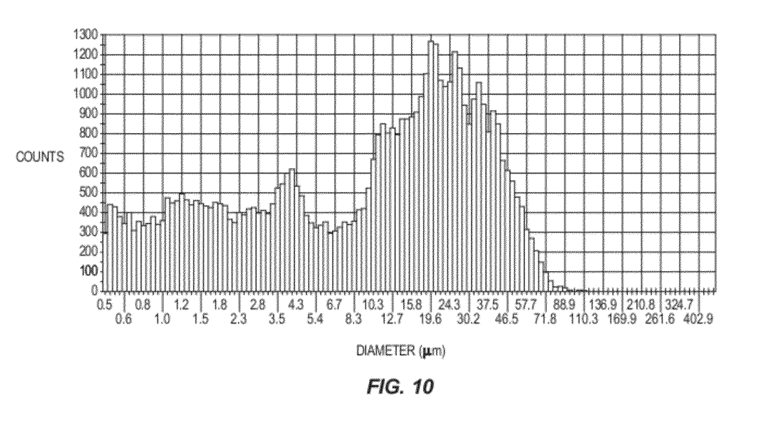

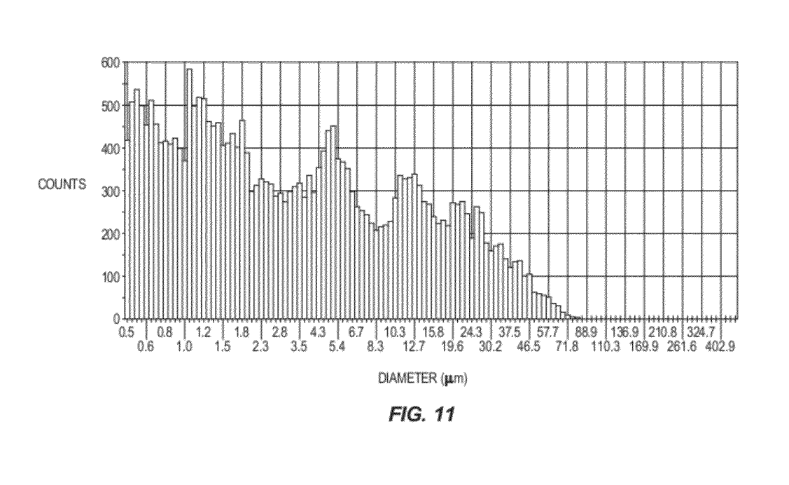

The water droplet size distribution for the initial mixture versus the mixture after filtration was analyzed using a particle counter. (See FIGS. 10 and 11). The results illustrated that the coalescer unit achieved ˜85.33% efficiency removal of water drops from the diesel fuel.

A water-oil coalescer with nanofiber coated springs may be used in other industrial applications, besides fuel water separation, such as in pharmaceuticals, specialty chemicals, petrochemicals, and other industries, where water drops are to be separated from oils. Through appropriate selection of fiber materials, it also is possible to reject oil drops from water, with applications in water filtration, oil spill cleanup, and environmental protection applications.

The nanofiber material of the nanofiber-coil coalescers may be polymeric fiber or other types of fiber. For example, the nanofibers may be ceramic fibers, metal oxide fibers, or carbon fibers.

The nanofiber material may be electro-spun and coated onto coil supporting structures. Alternatively, coarser fibers, such as melt-blown or melt-spun polymeric fibers may be coated onto supporting structures such as springs, cylindrical mesh or trellis materials, perforated tubes, foam tubes (e.g., a polyurethane or polyether foam tube which optionally may be hollow), and other porous supporting structures.

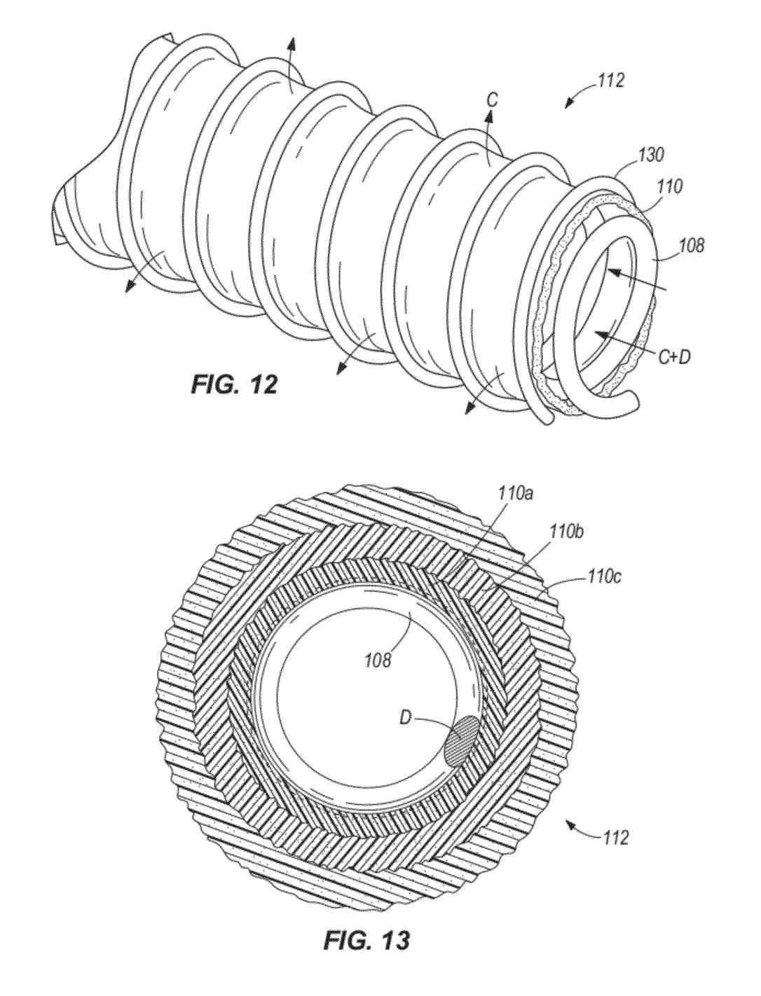

The disclosed nanofiber-coil coalescers may be utilized in systems where outside→inside flow is utilized to separate a dispersed phase from a continuous phase. Alternatively, the disclosed nanofiber-coil coalescers may be utilized in systems where inside→outside flow is utilized to separate a dispersed phase from a continuous phase provided that the flow rate is low enough that it does not damage the nanofiber layer or cause separation of the nanofiber layer from the coil. Alternatively, for higher flow rates with an inside to outside configuration, the nanofiber can be structurally supported and restrained by a surrounding wrap, wire, screen, glue, hot melt, other filter media, coil, spring, porous fabric, plastic mesh, or other means. FIGS. 12-15 illustrate coalescers configured for inside→outside flow. As shown in FIG. 12, the coalescer 112, includes a spring support structure 108, a nanofiber layer 110 on the outside of the spring support structure 108, and a surrounding coil 130, which prevents the nanofiber layer 110 from being displaced from the spring support structure 108 during use with inside→outside flow. In coalescers configured for inside→outside flow, the dispersed phase D, collects on the inside face of the nanofiber layer 110. FIGS. 13-15 illustrate collection of a drop of dispersed phase D on the inside face of the nanofiber layer 110.

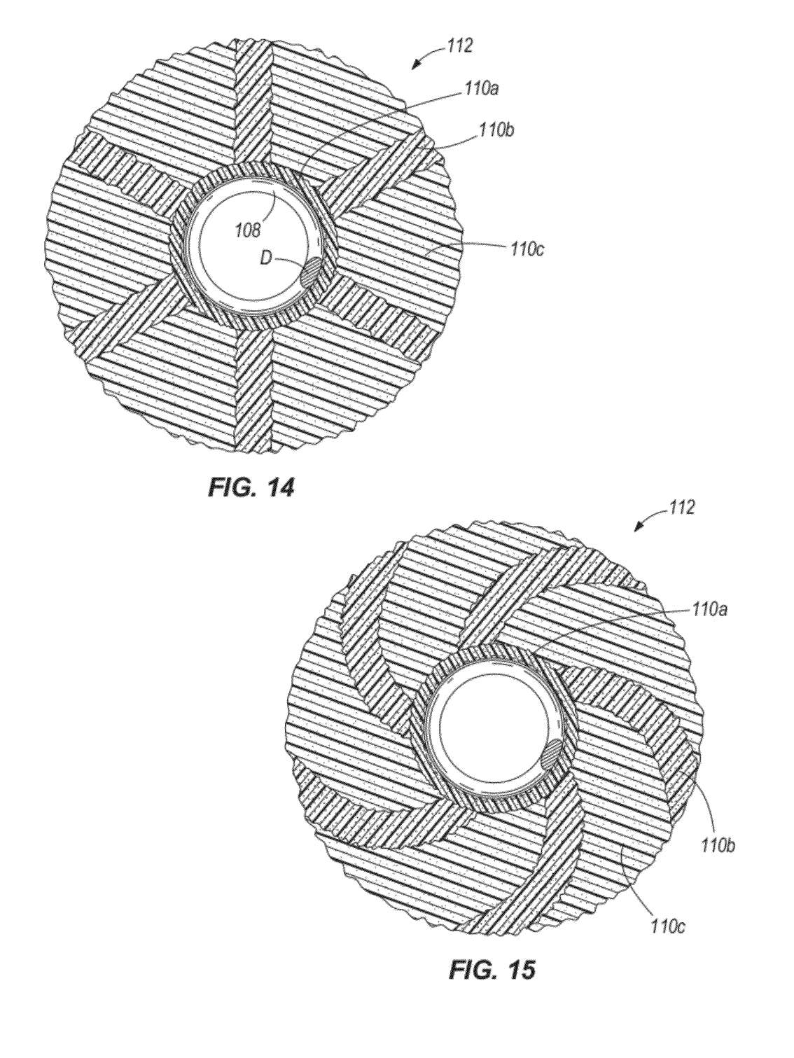

The disclosed coalescers may include a single layer of media material 110 or may include multiple composite layers of media having different properties (i.e., differing material, diameter, and/or orientation). (See FIGS. 13-15, 110a, 110b, and 110c). The layers can be applied such that their surfaces are oriented parallel to the surface of the tubular support structure. (See FIGS. 13, 110a, 110b, and 110c). Alternatively, the layers can also be applied such that they are perpendicular or angled with respect to the surface of the tubular support structure. (See FIGS. 14 and 15, 110a, 110b, and 110c).

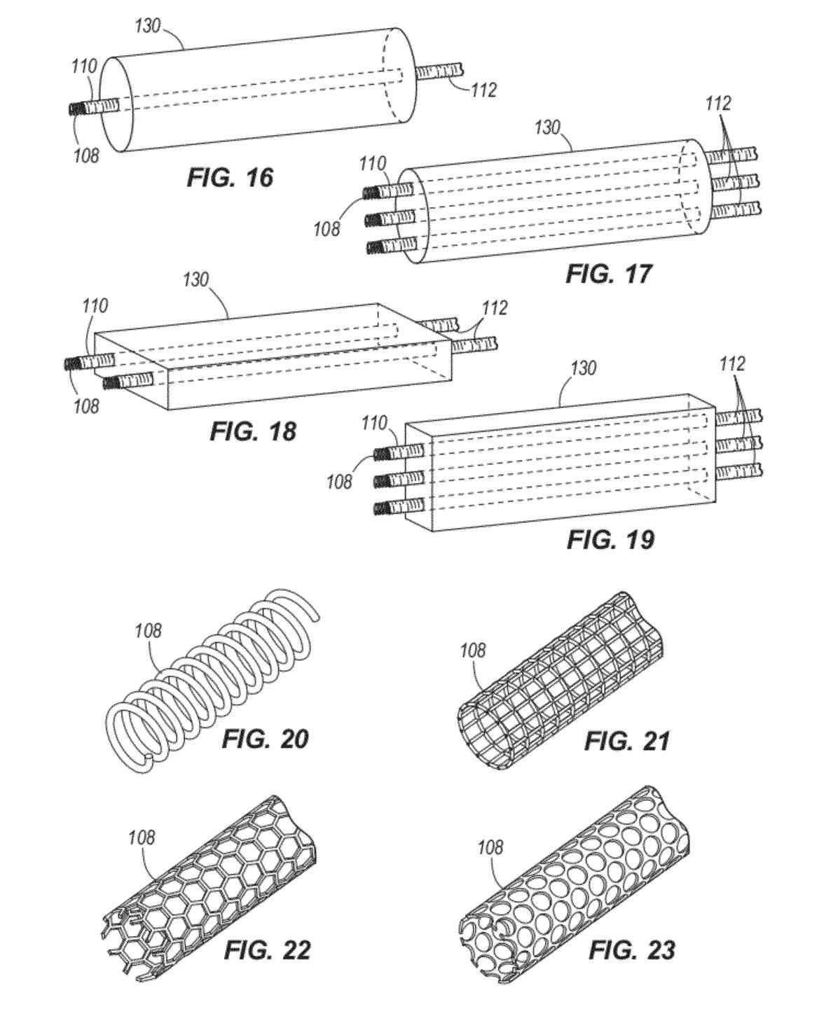

The disclosed coalescers may be utilized with additional media material. For example, as shown in FIGS. 16-19, one or more coalescers 112 may be mounted in additional media material 130.

The foregoing example utilized a spring as a porous tubular support structure. (See FIG. 20). However, other types of porous tubular support structures are contemplated, including mesh or trellis tubular structures (FIGS. 21 and 22) and perforated tubes (FIG. 23). Porous foam tubes, which optionally may include a hollow core, also are contemplated. In particular, solid, open-cell-structured foams, also known as reticulated foams) in tubular form are contemplated. In some embodiments, reticulated foam tubes having a density of about 0.5-10 lb/ft³ are contemplated.

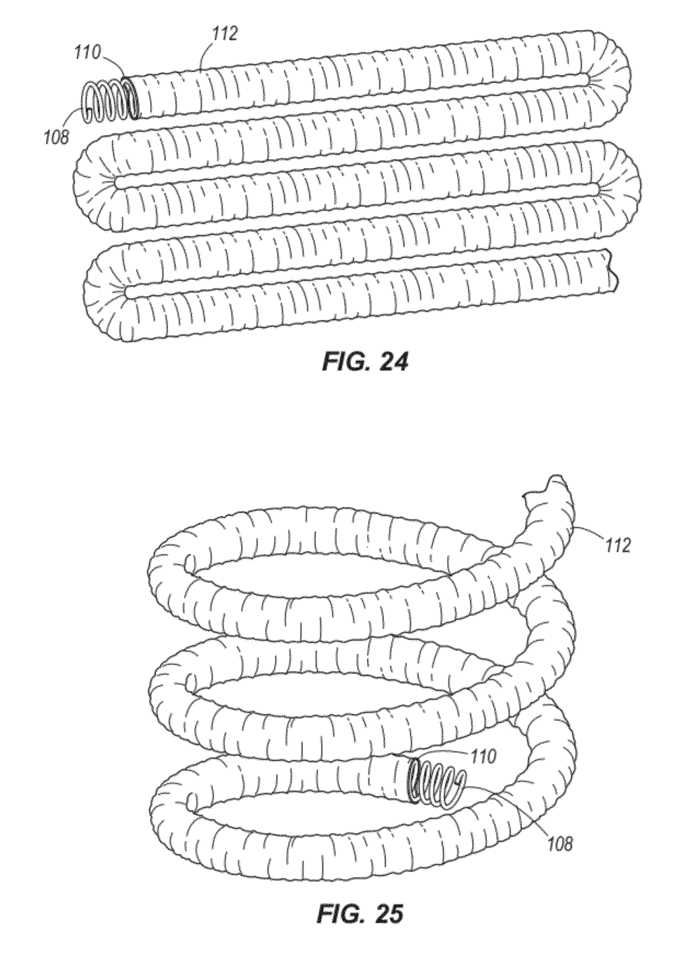

The coalescers disclosed herein may be formed into superstructures. In some embodiments, superstructures contemplated herein may include, but are not limited to, undulated tube superstructures (FIG. 24) and coiled tube superstructures (FIG. 25).

### REFERENCES

Keun-Hyung Lee, Osamu Ohsawa, Kei Watanabe, Ick-Soo Kim, Steven R. Givens, Bruce Chase, and John F. Rabolt, “Electro-spinning of syndiotactic polypropylene from a polymer solution at ambient temperatures,” Macromolecules, 42, 5215-5218, 2009.
Minglin Ma, Randal M. Hill, and Gregory C. Rutledge. “A review of recent results on super-hydrophobic materials based on micro- and nanofibers.” Journal of Adhesion Science and Technology (2008), 22(15), 1799-1817.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

What is claimed is:

1. A tubular surface coalescer comprising one or more layers of coalescing media material applied to an outer surface of a porous tubular support structure.

2. The tubular surface coalescer of claim 1, wherein the porous tubular support structure has a diameter of between about 1 mm and about 10 mm.

3. The tubular surface coalescer of claim 1, wherein the porous tubular support structure is a spring.

4. The tubular surface coalescer of claim 3, wherein the spring has an average distance between coils of about 0.5 mm and about 2.5 mm.

5. The tubular surface coalescer of claim 1, wherein the porous tubular support structure is a tubular mesh.

6. The tubular surface coalescer of claim 1, wherein the porous tubular support structure is a perforated hollow tube.

7. The tubular surface coalescer of claim 1, wherein the porous tubular support structure is a hollow tube.

8. The tubular surface coalescer of claim 1, wherein the coalescing media material comprises fibers having a mean diameter between about 0.05 μm and 5 μm.

9. The tubular surface coalescer of claim 8, wherein the fibers are polymeric fibers.

10. The tubular surface coalescer of claim 8, wherein the fibers are ceramic fibers.

11. The tubular surface coalescer of claim 8, wherein the fibers are carbon fibers.

12. The tubular surface coalescer of claim 8, wherein the fibers are applied to the outer surface of the porous tubular support structure via electro-spinning, melt-spinning, or melt-blowing.

13. The tubular surface coalescer of claim 1, wherein the coalescing media material has a mean pore size M, wherein 0.2 μm≦M≦12.0 μm.

14. The tubular surface coalescer of claim 1, wherein the media material has a maximum pore size M_{M} and 1≦M_{M}/M≦3.

15. The tubular surface coalescer of claim 1, comprising at least two adjacent layers of coalescing media material applied to the outer surface of the porous tubular support structure, namely an outer first layer of media material and an inner second layer of media material, and the first layer and the second layer having mean pore sizes M₁ and M₂, respectively, and M₁>M₂.

16. The tubular surface coalescer of claim 15, wherein M₁ is at least about 2.5 times greater than M₂.

17. The tubular surface coalescer of claim 15, wherein M₁≧30 μm.

18. The tubular surface coalescer of claim 15, wherein 0.2 μm≦M₂≦12.0 μm.

19. The tubular surface coalescer of claim 15, wherein the outer first layer of media material comprises media having an average fiber diameter that is less than about 100 μm.

20. The tubular surface coalescer of claim 15, wherein the coalescing media material is formed by electro-spinning, melt-spinning, or melt-blowing the inner second layer of media material on the porous tubular support structure, and subsequently electro-spinning, melt-spinning, or melt-blowing the outer first layer of media material on the inner layer of media material.

21. The tubular surface coalescer of claim 1, wherein the coalescer is flexible.

22. The tubular surface coalescer of claim 1, configured as a coiled tube.

23. The tubular surface coalescer of claim 1, configured as an undulating tube.

24. The tubular surface coalescer of claim 1 contained in a housing, the housing having an upstream inlet structured to receive a mixture and a downstream outlet structured to discharge the mixture after coalescing of the dispersed phase.

25. A coalescing system comprising one or more tubular surface coalescers of claim 1.

26. The coalescing system of claim 25, configured for removing water dispersed in hydrocarbon fuel.

27. The coalescing system of claim 25, wherein the system comprises one or more of the tubular surface coalescers aligned in parallel.

28. The coalescing system of claim 25, wherein the system has a flow rate determined by total number of the one or more tubular surface coalescers present in the system.

29. The coalescing system of claim 25, wherein the system has a flow rate determined by length of the one or more tubular surface coalescers present in the system.

30. A method of removing water drops dispersed in hydrocarbon fuel, the method comprising passing a mixture comprising hydrocarbon fuel and water dispersed in the hydrocarbon fuel through the tubular surface coalescer of claim 1 and removing at least about 90% of the water dispersed in the hydrocarbon fuel.

31. The method of claim 30, wherein the ratio of the diameter of the porous tubular support structure to the average diameter of the water drops dispersed in the hydrocarbon fuel is less than about 100.

32. A method of removing hydrocarbon fuel drops dispersed in water, the method comprising passing a mixture comprising water and hydrocarbon fuel dispersed in the hydrocarbon fuel through the tubular surface coalescer of claim 1 and removing at least about 90% of the hydrocarbon fuel dispersed in the water.

33. The method of claim 32, wherein the ratio of the diameter of the porous tubular support structure to the average diameter of the hydrocarbon drops dispersed in the water is less than about 100.

34. A method of removing hydrocarbon droplets dispersed in an airstream, the method comprising passing a mixture comprising an airstream and hydrocarbon mist dispersed in the airstream through the tubular surface coalescer of claim 1 and removing at least about 90% of the hydrocarbon mist dispersed in the airstream.

35. The method of claim 34, wherein the ratio of the diameter of the porous tubular support structure to the average diameter of the hydrocarbon droplets dispersed in the airstream is less than about 100.
